(19) United States
(12) Patent Application Publication
(10) Pub. No.: US 2006/0276462 A1
(43) Pub. Date: Dec. 7, 2006

(54) Method of treating cognitive decline due to sleep deprivation and stress

(76) Inventors: Sam A. Deadwyler, Winston-Salem, NC (US); Robert E. Hampson, Kernersville, NC (US); Linda J. Porrino, Winston-Salem, NC (US); Gary A. Rogers, Laguna Beach, CA (US); Gary S. Lynch, Irvine, CA (US)

Correspondence Address: COLEMAN SUDOL SAPONE, P.C., 714 COLORADO AVENUE, BRIDGE PORT, CT, 06605-1601, US

(21) Appl. No.: 10/541,687

(22) PCT Filed: Jan. 13, 2004
(86) PCT No.: PCT/US04/00706

§ 371 Date: Nov. 21, 2005

Related U.S. Application Data
(60) Provisional application No. 60/439,735, filed on Jan. 13, 2003.

(51) Int. Cl. A61K 31/538; C07D 413/02

(52) U.S. Cl. 514/229.5; 544/105

(57) ABSTRACT

This invention relates to methods of use for AMPA receptor potentiator compounds and pharmaceutical compositions in the prevention and treatment of cognitive impairment as a result of acute or chronic sleep deprivation, including enhancement of receptor functioning at synapses in brain networks responsible for higher order behaviors. A still further aspect of the present invention is the use of an active agent as described above for the preparation of a medicament for the treatment of a disorder as described above.

This invention has been supported by grant NIH DARPA (ARO) 43278-LS. The United States government retains certain rights in the invention.

### FIELD OF THE INVENTION

This invention relates to methods of use for compounds and pharmaceutical compositions in the prevention and treatment of cognitive impairment as a result of acute or chronic sleep deprivation, including enhancement of receptor functioning at synapses in brain networks responsible for higher order behaviors. A still further aspect of the present invention is the use of an active agent as described above for the preparation of a medicament for the treatment of a disorder as described above.

### BACKGROUND OF THE INVENTION

Sleep deprivation in humans is a critical problem in society. The human body requires 6-9 hours of sleep per day for optimum cognitive function. Total or partial loss of sleep impairs the ability to correctly process information and make appropriate decisions. Symptoms of sleep deprivation are similar to chronic stress. Sleep deprivation affects shift workers, mothers of newborns, long-distance drivers, personnel whose jobs require extended periods of wakefulness as well as people suffering from chronic sleep deprivation due to pain, illness, insomnia, sleep apnea, etc.

Electrophysiology of Sleep and Sleep-Deprivation

The electrophysiology of sleep has primarily been characterized by the frequency and power of the human EEG. During wakefulness, EEG activity varies widely in frequency and power, but is predominately low power, high frequency (>20 Hz) “alpha” activity. During sleep, activity in the 0.5-4.0 Hz “delta” band predominates in the initial non-REM or slow-wave sleep (SWS) period. During the sleep cycle, EEG frequency periodically increases during brief intervals of REM activity, then returns to the low frequency state. When the subject is drowsy, the EEG is characterized by increased spectral power of the delta frequency, and periods of activity similar to SWS (Gaudreau et al. 2001). This change in complexity of the EEG is also reflected in changes to event-related potentials such as P300, which is evoked by task-relevant stimuli. While performing a task, amplitude of P300 is inversely correlated with probability of stimulus occurrence. When the same stimuli are presented as the subject becomes drowsy and falls asleep, P300 decreases, and is replaced by two other evoked potentials, P220 and P900. The latter potentials exhibit a similar inverse correlation to stimulus probability as P300, but they are also inversely correlated to task relevance, suggesting a deficit in task-related processing in the drowsy state (Hull and Harsh, 2001).

Sleep deprivation produces increased 0.5-4.0 Hz and 18-25 Hz activity of the EEG (Gaudreau et al 2001), suggesting difficulty in maintaining wakefulness. Nonlinear analysis of the EEG also shows a reduction in high-order (i.e. complex) patterns during sleep-deprivation, which is thought to represent an alteration in information processing capability during sleep-deprivation (Jeong et al. 2001). A similar increase in low frequency spectral power and decreased complexity of neural activity is also observed in rodents during prolonged wakefulness (Schwierin et al. 1999). Likewise, there is increased low frequency activity, and SWS-like patterns following sleep deprivation (Ocampo-Garces et al. 2000; Huber et al. 2000).

Very little current research on sleep and sleep deprivation has been performed on nonhuman primates; however, similar patterns have been shown for human and monkey EEG. During alert wakefulness, the EEG is characterized by high frequency, low amplitude activity, while drowsy and sleep states show the same predominance of 0.5-4.0 Hz activity interspersed with episodic bouts of REM sleep (Reite et al. 1970). Following sleep deprivation, the waking EEG is marked by frequent periods of delta (0.5-4.0 Hz) and theta (8.0-12.0 Hz) as if the monkey were alternating between sleeping and waling states (David et al. 1975). A study of EEG frequencies while monkeys performed a delayed match to sample task in a “simulated spacecraft” demonstrated that correct performance was characterized by the high frequency, complex EEG patterns, while errors (particularly during drowsy periods) were characterized by low frequency EEG with increased coherence between recording sites (Berkhout et al. 1969).

Neuroanatomical Substrates of Sleep Deprivation

Although it has long been established that sleep deprivation interferes with the behavioral performance of a variety of tasks, including cognitive, motor, attention, and motivation, the neural substrates of these deficits remain unclear. Some of the most provocative evidence addressing these issues comes from studies in sleep-deprived humans utilizing non-invasive imaging methods. Studies with positron emission tomography (PET) have investigated changes in brain glucose metabolism accompanying sleep, sleep deprivation, and the effects of drugs to combat sleep deprivation. These methods are extremely powerful, allowing us to assess changes in brain function directly in living, conscious, behaving humans. However, there have been very few studies that have utilized these tools to investigate the neuroanatomical basis of sleep deprivation.

To investigate the effects of sleep deprivation, studies have been conducted in human populations largely comparing the patterns of brain functional activity that accompany task performance following normal sleep directly to those observed after sleep deprivation. Wu et al, (1991) employed positron emission tomography (PET) with [18F]-deoxyglucose (FDG) to measure rates of cerebral glucose utilization during a vigilance task. They found that sleep deprivation led to a significant reorganization of regional cerebral metabolic activity despite the fact that overall global rates of brain metabolism were not altered. Decreased metabolism was seen in the thalamus, basal ganglia and cerebellum during sleep deprivation compared to scans following normal sleep time. In addition, functional activity was decreased in temporal lobes and increased in visual cortex. The authors concluded that sleep deprivation dampens brain arousal mechanisms as reflected in the decreased metabolism in the basal ganglia and thalamus, whereas there are increased metabolic demands in areas related to the task, such as visual cortex vs. auditory systems. In addition, task performance was specifically correlated with glucose utilization in thalamus, caudate, putamen, and amygdala. Poor performance on the task was associated with the lowest rates of glucose utilization in these structures. These latter data imply that there is an important role of subcortical structures in determining the effects of sleep deprivation.

Other studies have largely confirmed these general findings of reorganization of functional activity following sleep deprivation (Braun et al., 1997). In one of the few studies in which a working memory task was utilized (Thomas et al., 1993), large decreases in cerebral metabolism were observed in the prefrontal cortex, particularly the orbitofrontal cortex, during performance of a task after sleep deprivation. Hence, many of these decreases were highly correlated to task performance. Thus, when working memory is necessary, the prefrontal cortex is an important element of the network of structures in which the effects of sleep deprivation are most apparent.

Another important approach has been the use of fMRI to study sleep deprivation. The results of these studies, although few in number, have also confirmed the idea that sleep deprivation results in a reorganization of brain functional activity. Comparisons of task performance were made following normal sleep and after sleep deprivation to identify the substrates of task performance during different states. However, one element that cannot be addressed by this strategy is the effect of sleep deprivation in general. The results of fMRI studies are expressly related to specific tasks and task performance only and do not address general effects of sleep deprivation or potential effects on other kinds of tasks or more particularly on mood and affect.

A critical factor identified by fMRI studies is the nature of the task. Distinct networks of brain structures appear to be involved following the performance of different tasks. The performance of working memory tasks involving verbal elements under conditions of sleep deprivation show increased activation within the prefrontal cortex and a lack of activation in the temporal cortex, as compared to performance of the same task following normal sleep (Drummond et al., 2000). In contrast, during the performance of an arithmetic task, the prefrontal cortex was activated in normal conditions of adequate sleep, but not during sleep deprivation conditions (Drummond et al., 1999). Studies in which an attention task was used showed that the difference between the constellation of structures activated during sleep deprivation conditions as compared to normal sleep conditions was focused in the thalamus (Portas et al., 1998). In other words, brain regions not specifically involved in task performance during normal conditions, were activated during sleep deprivation. The common element across these studies is clearly the reorganization of neural activity following sleep deprivation that can be attributed to the need for compensatory mechanisms to maintain task performance. There is recruitment of brain regions not normally involved in the performance of a specific task to compensate for the low arousal state consequent to sleep deprivation. There was a considerably higher amount of cognitive load involved in the verbal working memory task than in either the arithmetic or attention tasks. Working memory tasks may require the recruitment of the prefrontal cortex to a far greater degree than other tasks, and the degree of activation in prefrontal cortex may increase with higher working memory requirements.

Sleep deprivation has widespread effects on performance. Reviews of research in this area have concluded that the effects of sleep deprivation result in decreased reaction times, less vigilance, an increase in perceptual and cognitive distortions and changes in affect (cf. Krueger, 1989). A more recent study used a meta-analysis to provide a comprehensive analysis of the effects of sleep deprivation (Pilcher and Huffcutt, 1996). These authors analyzed 19 studies and concluded that mood is more affected by sleep deprivation than either cognitive or motor performance. These findings are consistent with the work of others in the field (Johnson, 1982; Koslowsky and Babkoff, 1992), in which it is clear that sleep deprivation produces significant increases in dysphoric mood. The changes in mood state that accompany sleep deprivation may result in non-specific depressive effects on brain functional activity. Effects not specific to cognitive performance need to be “subtracted out” from patterns of functional activity obtained during task performance. In addition, positive and negative affective states have been shown to correlate strongly with levels of dopamine in the striatum (cf. Volkow et al. 1999). This is of particular importance given the fact that the most effective wake-promoting compounds such as amphetamine and modafinil have been shown to act through dopaminergic systems Koob, 2000; Wisor et al., 2001).

All of the above studies that have utilized various imaging technologies have been conducted in human populations. Although they have the advantage of direct applicability, it can sometimes be difficult to assess the role of different environmental experience, sleep histories, educational history and experience with the tasks, as well as use of stimulant drugs such as caffeine and nicotine, potential psychiatric disorders, etc, all of which can affect the outcome. To isolate and identify the basic effects of sleep deprivation on brain functional activity, animal models are therefore important tools. Although considerable research has been conducted in rodent models, rodents have more limited behavioral repertoires and relatively poorly developed prefrontal cortex when compared to humans. Non-human primates as were employed here are, therefore, exceptional models in terms of their relevance to humans.

Role of Stress in Sleep Deprivation and Cognitive Performance

It is well recognized that chronic stress and/or glucocorticoids (GCs), such as corticosterone or cortisol (CORT), can negatively influence hippocampal-dependent cognition. Numerous studies have shown that chronic stress or CORT impairs learning and memory in animal models or in humans (Lathe, 2001; Porter et al., 2000; de Quervain et al., 1998; Lupien et al., 1998). Furthermore, studies have shown that chronic stress and/or CORT can impair hippocampal electrophysiology and accelerate age-related hippocampal anatomical changes in rodents (Porter et al., 2000; Porter, Landfield, 1998). Similar deleterious anatomical changes are found in hippocampus of primates (Sapolsky et al., 1990) and humans with elevated CORT (Cho, 2001; Lupien et al., 1998; Starkman et al., 1992). Thus, considerable evidence supports the view that chronic CORT accelerates the electrophysiological, anatomical and cognitive changes seen with aging, notably in hippocampus (Landfield, Eldridge, 1994; Porter, Landfield, 1998; Porter et al., 2000). This is of particular interest in the present context because extended sleep deprivation (ESD) also is a chronic stress that induces stress hormones (Spiegel et al., 1999; Suchecli et al., 1998). Moreover, ESD, and particularly rapid-eye movement sleep deprivation (REM-SD), disrupts memory consolidation and impairs cognitive performance much as does chronic stress (Graves et al., 2001). In addition, extraneous stressors can exacerbate the effects of prolonged SD (Suchecki et al., 1998). Together, the results suggest that ESD can be viewed as a form of chronic stress or a process exacerbated by stress hormones which accelerates brain aging.

Effects of Sleep-Deprivation on Memory

The effects of sleep-deprivation have been shown to include impairment of a subject's ability to concentrate, attend to relevant stimuli, and make appropriate discriminations between stimuli—i.e. to perform complex mnemonic tasks, which current research suggests are dependent on the hippocampus. It has been shown that during slow-wave sleep, the mammalian hippocampus appears to reactivate neurons in a manner similar to neural activity patterns recorded while the animal actively explored its environment immediately prior to the sleep period (Pavlides & Winson, 1989; Wilson & McNaughton 1994). Multiple sleep periods are necessary for short-term, hippocampal-dependent memories to become consolidated to long-term memory (Kim & Fanselow, 1992). Likewise, sleep deprivation impairs memory performance in learned avoidance (Bueno et al. 1994), water maze (Smith and Rose, 1996) and radial maze tasks (Smith et al. 1998)—each of which involves the hippocampus for learning and correct behavioral performance. Sleep deprivation causes increased serotonin metabolism (Youngblood et al. 1999), reduced norepinephrine (Porkka-Heiskanen et al. 1995), and an increase in prostaglandin (PGE2) synthesis (Moussard et al. 1994).

The Role of the Hippocampus in Memory

To more closely model the effects of sleep deprivation on a human subject's performance requires a well-learned behavioral task in which the cognitive processing of stimuli (e.g. working memory) can be assessed, separate from decreased ability to behaviorally perform the task. The mammalian hippocampus has been implicated in many behavioral tasks in which a subject must process or encode information about a stimulus, retain that information over a period of time, and perform a behavioral response appropriate to the “remembered” features of the stimulus. The role of the hippocampus in memory has been developed over many years with reports showing memory deficits in humans following damage to the medial temporal lobe and hippocampus (Scoville and Milner, 1957; Zola-Morgan et al., 1986; Squire et al., 1988; Squire and Cave, 1991). Although there has been continual refinement of theories of hippocampal function, it is now accepted that lesions of hippocampus and associated areas impair spatial working memory (Angeli et al., 1993; Cho et al., 1993), as well as nonspatial memory in a spatial task (Hampson et al. 1999a; Eichenbaum et al., 1992; Eichenbaum et al., 1994). It has become apparent from lesion studies that the hippocampus is essential to representing not just position, but relationships between stimuli (especially spatial stimuli), and that the projections between hippocampus and retrohippocampal areas are essential to the memory storage of these representations (Otto et al., 1991; Leonard et al., 1995;) and hence to the decision process required by the behavioral task.

Hippocampal Behavioral/Electrophysiological Model of Performance

Recordings of multiple single neurons in mammalian hippocampus during a short-term, working memory task have shown a dependence of behavioral performance on the hippocampal neural activity. In recent studies, different neural correlates of behavioral events during a spatial delayed-nonmatch-to-sample task have been identified (Deadwyler et al. 1996). These “functional cell types” show differential firing in response to specific classifications of behavioral events and represent a hierarchical encoding of critical features of the task (Hampson et al. 1 999b). It has also shown that the strength of this encoding can be used to predict different types of behavioral errors prior to their occurrence in the task (Haampson & Deadwyler 1996; Hampson et al. 1998a,b). This task has recently been developed for use in nonhuman primates as described below (FIGS. 1-3).

The present invention provides a means of overcoming the effects of sleep deprivation in circumstances that simulate cognitive demands in humans engaged in complex tasks. The described invention will attenuate and potentially alleviate the deleterious effects of sleep deprivation in nonhuman and human primates engaged in tasks requiring precise motor responses based on short-term memory. The invention identifies, in the same nonhuman primates, those brain regions that are altered during prolonged sleep deprivation, using electrophysiological recording techniques coupled with noninvasive imaging methods. These unique features of the invention will become evident from the following description.

The primary testing component (Component 1) used in the present invention consists of a nonhuman primate model that employs many-neuron recording techniques to assesses changes in identified neural ensemble correlates of short-term memory and motor performance during sleep deprivation. In Component 2, parallel assessment and identification of regional brain metabolic changes following sleep deprivation, utilizing Positron Emission Tomography (PET), was conducted in the same nonhuman primates, providing a complementary approach for determining key brain areas susceptible to change during sleep deprivation.

The two components of the present invention are listed below:

Component 1: Behavioral/electrophysiological model of short-term memory and motor performance in nonhuman primates. This component employed a currently in-use model of information processing during a delayed-match-to-sample (DMS) short term memory task, utilizing nonhuman primates. Several neuronal correlates of performance accuracy in this task have been obtained with custom designed multiple, single-cell recordings of neuron ensembles from hippocampus, striatum and somatosensory cortex. Sleep deprivation was varied and the animal tested during different periods of the day/night cycle. Specific patterns in hippocampal neuronal firing were identified that correlate to success or failure in performance of the task. Eye and limb movement tracking was employed to monitor attention to the task and ability to complete the behavioral response requirements.

Component 2: Imaging correlates of sleep deprivation in nonhuman primates. Recently adapted noninvasive neuroimaging techniques for nonhuman primates utilizing “micro” positron emission tomography (MicroPET) were used to examine neural activity in different brain regions in the same subjects tested in Component 1. The uniqueness of this approach is that repeated PET scans can be obtained from the same animals over time using the same metabolic markers utilized in humans. Simultaneous imaging and electrophysiological measurements allowed direct correlation of these measures to performance changes produced by sleep deprivation obtained in Component 1.

In Phase I the present invention utilized state of the art electrophysiological recording, imaging and analysis techniques to identify and target changes in critical brain regions involved in performance during prolonged periods of sleep deprivation in nonhuman primates. In Phase II, compounds that are known AMPA receptor positive modulators (potentiators; also known as Ampakines) were used to ameliorate the decline in cognitive function resulting from sleep deprivation.

### BRIEF DESCRIPTION OF THE DRAWINGS

FIG. 1 discloses the effect of 1-(benzofurazan-5-ylcarbonyl)morpholine (BCM) on the cognitive performance of sleep deprived non-human primates in a Delayed Match To Sample (DMTS) task. More specifically, the DMTS task revealed that sleep deprivation caused a marked decrease in cognitive performance, which was completely reversed by the administration of 0.8 mg/kg of BCM.

FIG. 2 discloses the effect of 1-(benzofurazan-5-ylcarbonyl)morpholine (BCM) on the absolute, regional metabolic activity of sleep deprived non-human primates engaged in a Delayed Match To Sample (DMTS) task as revealed by positron emission tomography using FDG. The difference between the uptake of FDG by sleep deprived subjects during the task vs baseline is contrasted to the difference when treated with BCM.

FIG. 3 discloses the data of FIG. 2 normalized to global metabolism under the three conditions of baseline, sleep deprivation and sleep deprivation treated with BCM. These data compare the effects of sleep deprivation on baseline uptake of FDG to the effects of BCM administration on uptake following sleep deprivation.

### DETAILED DESCRIPTION OF THE INVENTION

### Definitions

Unless defined otherwise, all technical and scientific terms used herein have the same meaning as commonly understood by those of ordinary skill in the art to which this invention belongs. For purposes of the present invention, the following terms are defined below.

“Subjects” or “patients” herein are generally mammalian (dogs put in contemplated uses also as well as humans) and more particularly human subjects. The subjects or patients may be male or female and may be at any stage of development, including adolescent, adult, geriatric (aged), etc., with adult subjects being preferred.

“AMPA receptor modulators” as used herein and as further described in any number of patents/applications referenced herein, are pharmacologic agents that act on the AMPA subtype of glutamate receptors located on neurons and glial cells in the brain and CNS of a subject or patient. Positive AMPA receptor modulators (synonymously, “AMPA receptor potentiators or up-modulators”) alter the functional properties of the AMPA receptor, consequently enhancing glutamatergic neurotransmission between neurons and thus facilitating cognitive function when this occurs in critically relevant brain regions. AMPA receptor modulators have been shown to increase neural activity and to improve cognitive performance in animal (rodents and nonhuman primates) tasks that require both “short-term retention” and “working memory.”

The term “treat” as used herein refers to any type of treatment that imparts a benefit to a patient afflicted with a dysfunction, including improvement in the condition of the subject (e.g., in one or more symptoms), etc.

The term “acute” as used herein refers to a short-term condition in which no substantial physiological adaptation within the subject or patient occurs. An “acute” condition may be one lasting less than 1 or 2 days, depending upon the particular situation.

The term “chronic” as used herein refers to a longer-term condition in which physiological adaptation within the subject or patient occurs. A “chronic” condition is not an “acute” condition. A “chronic” condition may be one lasting more than 2 or 3 days, depending upon the particular situation.

The phrase “concurrent administration,” as used herein refers to two active compounds that are administered at the same point in time (i.e, “simultaneous administration”), or sufficiently close in time so that the results of the two compounds together achieve a combined effect in the subject or patient.

The term “effective” as used herein refers to an amount of an agent, compound or pharmaceutical composition which produces an intended effect within the context of its use.

The term “prevention” within context shall mean “reducing the likelihood” or preventing a condition or disease state from occurring as a consequence of administration or concurrent administration of one or more compounds or compositions according to the present invention, alone or in combination with another agent.

This invention is directed to methods of using effective amounts of AMPA receptor potentiator compounds and pharmaceutical compositions in the prevention and treatment of cognitive impairment as a result of acute or chronic sleep deprivation, including enhancement of receptor functioning at synapses in brain networks responsible for higher order behaviors. A still further aspect of the present invention is the use of an active agent as described above for the preparation of a medicament for the treatment of a disorder as described above.

This invention is also directed to a pharmaceutical composition for use in the treatment or prevention of cognitive impairment as a result of acute or chronic sleep deprivation in a patient or subject, comprising an effective amount of an AMPA receptor potentiator in combination with a pharmaceutically acceptable, carrier, additive or excipient.

This invention is also directed to the use of a composition in the manufacture of a medicament for treating or preventing cognitive impairment as a result of acute or chronic sleep deprivation in a patient or subject, said composition comprising an effective amount of an AMPA receptor potentiator in combination with a pharmaceutically acceptable carrier, additive or excipient.

Compounds that enhance the functioning of the AMPA form of glutamate receptors facilitate the induction of LTP and the acquisition of learned tasks as measured by a number of paradigms. See, for example, Granger et al., Synapse 15:326-329 (1993); Staubli et al., PNAS 91:777-781 (1994); Arai et al., Brain Res. 638:343-346 (1994); Staubli et al., PNAS 91:11158-11162 (1994); Shors et al., Neurosci. Let. 186:153-156 (1995); Larson et al., J. Neurosci. 15:8023-8030 (1995); Granger et al., Synapse 22:332-337 (1996); Arai et al., JPET 278:627-638 (1996); Lynch et al., Internat. Clin. Psychopharm. 11: 13-19 (1996); Lynch et al., Exp. Neurology 145:89-92 (1997); Ingvar et al., Exp. Neurology 146:553-559 (1997); Hampson, et al., J. Neurosci. 18:2748-2763 (1998); and Lynch and Rogers, U.S. Pat. No. 5,747,492. There is a considerable body of evidence showing that LTP is a substrate of memory. For example, compounds that block LTP interfere with memory formation in animals, and certain drugs that disrupt learning in humans antagonize the stabilization of LTP, as reported by del Cerro and Lynch, Neuroscience 49: 1-6 (1992). Examples of suitable AMPA receptor up-modulators/potentiators (Ampakines) useful for the practice of the present invention include, but are not limited to those disclosed in:

U.S. Pat. No. 5,650,409 issued Jul. 22, 1997;

U.S. Pat. No. 5,736,543 issued Apr. 7, 1998;

U.S. Pat. No. 5,747,492 issued May 5, 1998;

U.S. Pat. No. 5,783,587 issued Jul. 21, 1998;

U.S. Pat. No. 5,852,008 issued Dec. 22, 1998;

U.S. Pat. No. 5,891,871 issued Apr. 6, 1999;

U.S. Pat. No. 5,962,447 issued Oct. 5, 1999;

U.S. Pat. No. 5,985,871 issued Nov. 16, 1999;

U.S. Pat. No. 6,110,935 issued Aug. 29, 2000;

U.S. Pat. No. 6,124,278 issued Sep. 26, 2000;

U.S. Pat. No. 6,274,600 issued Aug. 14, 2001;

U.S. Pat. No. 6,313,115 issued Nov. 6, 2001

U.S. Pat. No. 6,174,922 issued Jan. 16, 2001;

U.S. Pat. No. 6,303,816 issued Oct. 16, 2001;

U.S. Pat. No. 6,355,655 issued Mar. 12, 2002;

U.S. Pat. No. 6,358,981 issued Mar. 19, 2002;

U.S. Pat. No. 6,358,982 issued Mar. 19, 2002;

U.S. Pat. No. 6,362,230 issued Mar. 26, 2002;

U.S. Pat. No. 6,387,954 issued May 14, 2002;

U.S. Pat. No. 6,500,865 issued Dec. 31, 2002;

U.S. Pat. No. 6,515,026 issued Feb. 4, 2003;

U.S. Pat. No. 6,521,605 issued Feb. 18, 2003;

U.S. Pat. No. 6,525,099 issued Feb. 25, 2003;

U.S. Pat. No. 6,552,086 issued Apr. 22, 2003;

U.S. Pat. No. 6,596,716 issued Jul. 22, 2003;

U.S. Pat. No. 6,617,351 issued Sep. 9, 2003;

U.S. Pat. No. 6,639,107 issued Oct. 28, 2003;

WO 94/02475 published Feb. 3, 1994;

WO 96/38414 published Dec. 5, 1996;

WO 97/34878 published Sep. 25, 1997;

WO 97/36907 published Oct. 9, 1997;

WO 98/33496 published Aug. 6, 1998;

WO 98/12185 published Mar. 26, 1998;

WO 98/35950 published Aug. 20, 1998;

WO 99/33469 published Jul. 8, 1999;

WO 99/42456 published Aug. 26, 1999;

WO 99/43285 published Sep. 2, 1999;

WO 99/44612 published Mar. 2, 1999;

WO 99/51240 published Oct. 14, 1999;

WO 00/06083 published Feb. 10, 2000;

WO 00/06148 published Feb. 10, 2000;

WO 00/06149 published Feb. 10, 2000;

WO 00/06156 published Feb. 10, 2000;

WO 00/06157 published Feb. 10, 2000;

WO 00/06158 published Feb. 10, 2000;

WO 00/06159 published Feb. 10, 2000;

WO 00/06176 published Feb. 10, 2000;

WO 00/06537 published Feb. 10, 2000;

WO 00/06539 published Feb. 10, 2000;

WO 00/66546 published Nov. 9, 2000;

WO 00/75123 published Dec. 14, 2000;

WO 01/42203 published Jun. 14, 2001;

WO 01/57045 published Aug. 9, 2001;

WO 01/68592 published Sep. 20,2001;

WO 01/90056 published Nov. 29, 2001;

WO 01/90057 published Nov. 29, 2001;

WO 01/94306 published Dec. 13, 2001;

WO 01/96289 published Dec. 20, 2001;

WO 02/14275 published Feb. 21, 2002;

WO 02/14294 published Feb. 21, 2002;

WO 02/18329 published Mar. 7, 2002;

WO 02/32858 published Apr. 25, 2002;

WO 02/089734 published Nov. 14, 2002;

WO 02/098846 published Dec. 12, 2002;

WO 02/098847 published Dec. 12, 2002;

WO 03/045315 published Jun. 5, 2003; the disclosures of which are all hereby incorporated by reference. The above suitable AMPA receptor potentiators are readily prepared by one of ordinary skill in the art following, for example, the procedures set forth therein.

Specific examples of suitable AMPA receptor potentiators are listed in Table 1.

TABLE 1
Suitable AMPA Receptor Potentiators

| Example | Compound |
|---|---|
| 1 | 1-(Benzofurazan-5-ylcarbonyl)morpholine (BCM) |
| 2 | 1-(Quinoxaline-6-ylcarbonyl)piperidine (CX516). |
| 3 | 2H,3H,6aH-Pyrrolidino[2″,1″-3′,2′]1,3- |
| | oxazino[6′,5′-5,4]benzo[e]1,4-dioxan-10-one (CX614) |
| 4 | 3aH,9aH-pyrrolidino[2,1-b]pyrrolidino[2″,1″- |
| | 2′,3′](1,3-oxazino)[5′,6′-2,1]benzo[4,5-e]1,3- |
| | oxazaperhydroine-6,12-dione |
| | (Example 1 of PCT Patent Application No. US02/37646, |
| | filed Nov. 25, 2002) |
| 5 | The active single isomer of Example 4 |
| 6 | Aniracetam |
| 7 | IDRA-21 |
| 8 | S18986 |
| 9 | PEPA |
| 10 | [2-Fluoro-2-(4-{3-[(methylsulfonyl)amino]phenyl}propyl] |
| | [(methylethyl)sulfonyl]amine (Example 4 of WO 01/89510) |
| 11 | The active single enantiomer of Example 9 |
| 12 | N-2-(4-(3-thienyl)phenyl)propyl methanesulfonamide |
| | (Example 5 of WO 98/33496) |
| 13 | LY392098 |
| 14 | LY404187 |
| 15 | LY450108 |
| 16 | LY451398 |

Specific structures of suitable AMPA receptor up-modulators are illustrated below: 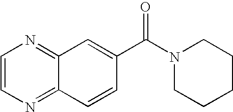
1-(quinoxaline-6-ylcarbonyl)piperidine (CX516) 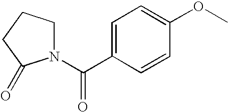
Aniracetam 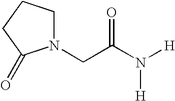
Piracetam 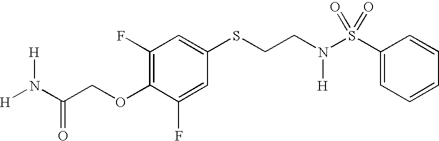
PEPA; 2-(2,6-difluoro-4-{2-[(phenylsulfonyl)amino]ethylthio}phenoxy)acetamide 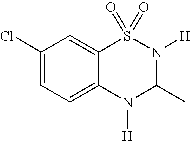
IDRA-21; 7-chloro -3-methyl-2H,3H,4H-benzo[e]1,2,4-thiadiazaperhydroine-1,1-dione 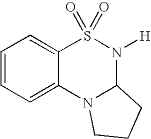
(S)-2,3-dihydro-[3,4]cyclopentano-1,2,4-benzothiadiazine-1,1-dioxide: (S18986-1) 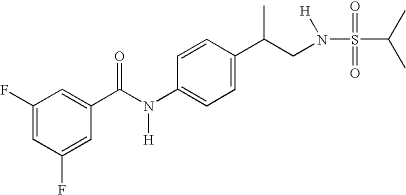
N-[4-(1-methyl-2-{[(methylethyl)sulfonyl]amino}ethyl)phenyl](3,5-difluorophenyl)carboxamide
N-[4-((1R)-1-methyl-2-{[(methylethyl)sulfonyl]amino}ethyl)phenyl](3,5-difluorophenyl)carboxamide (LY450108)
N-[4-((1S)-1-methyl-2-{[(methylethyl)sulfonyl]amino}ethyl)phenyl](3,5-difluorophenyl)carboxamide 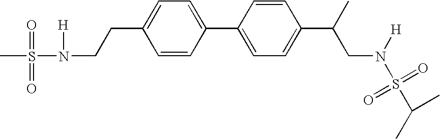
(2-{4-[4-(1-methyl-2-{[(methylethyl)sulfonyl]amino}ethyl)phenyl]phenyl}ethyl)(methylsulfonyl)amine
(2-{4-[4-((1R)-1-methyl-2-{[(methylethyl)sulfonyl]amino}ethyl)phenyl]phenyl}ethyl)(methylsulfonyl)amine (LY451395)
(2-{4-[4-((1S)-1-methyl-2-{[(methylethyl)sulfonyl]amino}ethyl)phenyl]phenyl}ethyl)(methylsulfonyl)amine 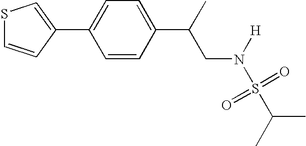
[(methylethyl)sulfonyl][2-(4-(3-thienyl)phenyl)propyl]amine (LY392098) 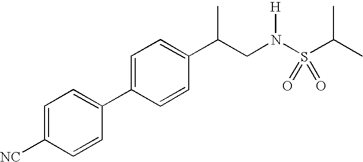
4-[4-(1-methyl-2-{[(methylethyl)sulfonyl]amino}ethyl)phenyl]benzenecarbonitrile (LY404187) 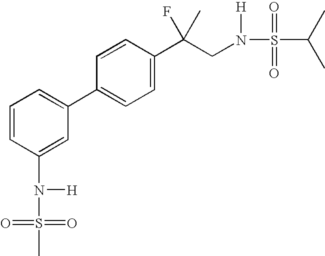
[2-fluoro-2-(4-{3-[(methylsulfonyl)amino]phenyl}phenyl)propyl][(methylethyl)sulfonyl]amine
[(2S)-2-fluoro-2-(4-{3-[(methylsulfonyl)amino]phenyl}phenyl)propyl][(methylethyl)sulfonyl]amine (LY503430)
[(2R)-2-fluoro-2-(4-{3-[(methylsulfonyl)amino]phenyl}phenyl)propyl][(methylethyl)sulfonyl]amine.

The series of compounds represented by the figure 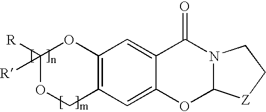
wherein

Z are is —CH₂— or

R and R′ are independently hydrogen, alkyl, substituted alkyl or together form a cycloalkyl ring, or together with oxygen, sulfur or nitrogen form a heterocyclic ring.

m is 0, 1 or 2, and,

n is 1 or 2.

More preferred are compounds such as 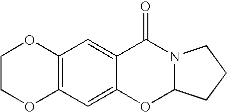
2H,3H,6aH-pyrrolidino[2″,1″-3′,2′]1,3-oxazaperhydroino[6′,5′-2,1]benzo[4,5-e]1,4-dioxin-10-one (CX614), or 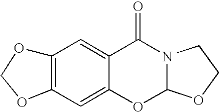
2H,7H,8H,5aH-1,3-oxazolidino[2″,3″-3′,2′]1,3-oxazaperhydroino[6′,5′-4,5]benzo[d]1,3-dioxolen-9-one (CX554), or 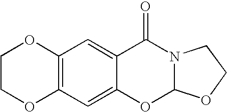
2H,3H,8H,9H,6aH-1,3-oxazolidino[2″,3″-3′,2′]1,3-oxazaperhydroino[6′,5′-4,5]benzo[e]1,4-dioxin-10-one.

The series of compounds represented by the figure 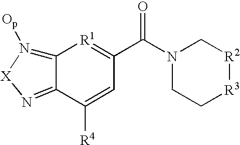
wherein X=oxygen or sulfur; R¹ is selected from the group consisting of —N═, —CR═, or —CX═; R² is selected from the group consisting of —(CRR′)ₙ—, —C(O)—, —CR═CR′—, —CR═CX—, —CRX—, —CXX′—, —S—, and —O—, and R³ is selected from the group consisting of —(CRR′)ₘ—, —C(O)—, —CR═CR′—, —CRX—, —CXX′—, —S—, and —O—; R⁴ is R or X; X and X′ are independently selected from —Br, —Cl, —F, —CN, —NO₂, —OR, —SR, —NRR′, —C(O)R, —CO₂R, or —CONRR′, wherein two groups R or R′ on an individual group X, or on two adjacent groups X, may together form a ring; and

R and R′ are independently selected from (i) hydrogen, (ii) C₁-C₆ branched or unbranched alkyl, which may be unsubstituted or substituted with one or more functionalities selected from halogen, nitro, alkoxy, hydroxy, alkylthio, amino, keto, aldehyde, carboxylic acid, carboxylic ester, or carboxylic amide, and wherein two such alkyl groups on a single carbon or on adjacent carbons may together form a ring, and (iii) aryl, which may be unsubstituted or substituted with one or more functionalities selected from halogen, nitro, alkoxy, hydroxy, aryloxy, alkylthio, amino, keto, aldehyde, carboxylic acid, carboxylic ester, or carboxylic amide;

m and p are, independently, 0 or 1; and n is 0, 1 or 2.

Preferred examples in this series of compounds are: 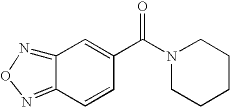
1-(benzofurazan-5-ylcarbonyl)piperidine 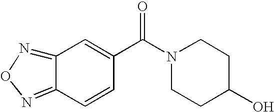
1-(benzofurazan-5-ylcarbonyl)-4-hydroxypiperidine 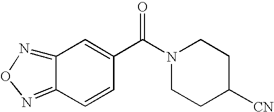
1-(benzofurazan-5-ylcarbonyl)-4-cyanopiperidine 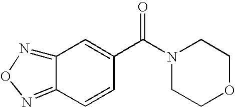
1-(benzofurazan-5-ylcarbonyl)morpholine (BCM) 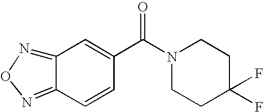
1-(benzofurazan-5-ylcarbonyl)-4,4-difluoropiperidine.

The series of compounds represented by the figure 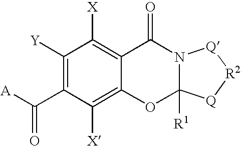
wherein

Q and Q′ are independently hydrogen, —CH₂—, —O—, —S—, alkyl, or substituted alkyl,

R¹ is hydrogen or alkyl,

R² may be absent, or if present may be —CH₂—, —CO—, —CH₂CH₂—, —CH₂CO—, —CH₂O—, —CRR′—, or —CONR—,

Y is hydrogen or —OR³, or serves to link the aromatic ring to A as a single bond, ═N— or —NR—,

R³ is hydrogen, alkyl, substituted allyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,

A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,

R is hydrogen, aryl, arylalkyl, substituted aryl, substituted arylalkyl, alkyl, substituted alkyl, cycloalkyl, substituted cycloalkyl, or heterocycloalkyl,

R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,

X and X′ are independently R, halo, —CO₂R, —CN, —NRR′, —NRCOR′, —NO₂, —N₃ or —OR.

Preferred examples in this series of compounds are wherein

Q, Q′ and R² are —CH₂—,

X, X′ and R¹ are hydrogen,

Y is hydrogen or —OR³, where R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,

A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,

R is hydrogen, aryl, arylalkyl, substituted aryl, substituted arylalkyl, alkyl, substituted allyl, cycloalkyl, substituted cycloalkyl, or heterocycloalkyl,

R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,

X and X′ are independently R, halo, —CO₂R, —CN, —NRR′, —NRCOR′, —NO₂, —N₃ or —OR.

Other preferred examples are wherein

Q and Q′ are independently hydrogen, alkyl, or substituted alkyl,

R¹ is hydrogen or allyl,

R² is absent,

Y is hydrogen or —OR³, or serves to link the aromatic ring to A as a single bond, ═N— or —NR—,

R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,

A is —NRR′, —OR, alkyl, substituted alkyd, cycloalkyl, substituted cycloalkyl, cycloalkylalkyl, aryl, substituted aryl, a hetero cycle or a substituted hetero cycle containing one or two heteroatoms such as oxygen, nitrogen or sulfur,

R is hydrogen, aryl, arylalkyl, substituted aryl, substituted arylalkyl, alkyl, substituted alkyl, cycloalkyl, substituted cycloalkyl, or heterocycloalkyl,

R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,

X and X′ are independently R, halo, —O₂R, —N, —NRR′, —NRCOR′, —NO₂, —N₃ or —OR.

Yet other preferred examples are wherein

Q and Q′ are independently hydrogen, alkyl, or substituted alkyl,

R¹ is hydrogen or alkyl,

R² is absent,

Y is —OR³,

R³ is a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,

A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms, such as oxygen, nitrogen or sulfur,

R is hydrogen, aryl, arylalkyl, substituted aryl, substituted arylalkyl, alkyl, substituted alkyl, cycloalkyl, substituted cycloalkyl, or heterocycloalkyl,

R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,

X and X′ are independently R, halo, —CO₂R, —CN, —NRR′, —NRCOR′, —NO₂, —N₃ or —OR.

Especially preferred examples in this series of compounds are:

Q and Q′ are independently hydrogen, alkyl, or substituted alkyl,

R¹ is hydrogen or alkyl,

R² is absent,

Y is —OR³,

R³ is a lower alkylene such as a methylene or ethylene, or substituted lower alkylene such as —CRR′— linking the aromatic ring to A to form a substituted or unsubstituted 6, 7 or 8-membered ring,

A is —NRR′, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,

R is hydrogen, aryl, arylalkyl, substituted aryl, substituted arylalkyl, alkyl, substituted alkyl, cycloalkyl, substituted cycloalkyl, or heterocycloalkyl,

R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,

X and X′ are independently R, halo, —CO₂R, —CN, —NRR′, —NRCOR′, —NO₂, —N₃ or —OR.

Especially preferred examples are 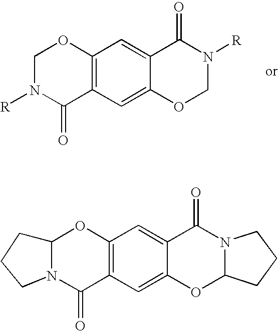
3aH,9aH-pyrrolidino[2,1-b]pyrrolidino[2″,1″-2′,3′](1,3-oxazino)[5′,6′-2,1]benzo[4,5-e]1,3-oxazaperhydroine-6,12-dione,

There are several novel aspects to the present invention; the first is in the use of nonstimulants to counter effects of sleep deprivation. Currently accepted means of improving attention to task and wakefulness include caffeine and amphetamines. Both of these are stimulants that have effects throughout the body, not just in the brain. In addition, there is addictive and abuse potential to the use of stimulants. Thus, there is a need for improved treatments that lack the inherent side effect liability that stimulants present. Second, this is a novel use for positive AMPA receptor modulators. This present invention proposes that positive AMPA receptor modulators be used to counter the cognitive decline that results from sleep deprivation. AMPA receptor modulators can be given at any time during the state of sleep deprivation to improve cognitive performance.

While the following list is not meant to be limiting in any way, those contemplated as benefiting from the practice of the present invention are:
1. Persons or other mammals with circadian rhythm disruption such as, but not limited to: a) shift workers who must alter their activities from day to night or vice-versa, and hence encounter sleep loss due to disruption of sleep cycle, b) persons on extended work assignments such as pilots, health care workers or service animals, for whom continual alertness (and consequent loss of sleep) is essential to their task or their personal safety, or c) persons who travel quickly through multiple time zones and must perform cognitive tasks before they are fully adjusted to the new zone (jet-lag).
2. Caretakers of newborns/invalids/critically ill patients: individuals who must awaken frequently during the night to care for another person.
3. Patients: those with disease states that disrupt sleep, such as insomnia, sleep apnea, chronic pain, etc.
4. Persons who voluntarily extend their waking period beyond normal limits such that the resulting loss of sleep causes a cognitive decline.
   I. Biological Activity

Several methods can be used in order to determine whether a particular compound has the ability to potentiate AMPA receptor function.

A. Enhancement of AMPA Receptor Function in Dissociated Neurons Using Whole-Cell Patch-Clamp Techinque.

Cortical/Hippocampal cells are prepared from day 18-19 embryonic Sprague-Dawley rats and recorded after 3/4 to 7/8 days in culture. The following solutions are used: extracellular solution/saline (in my: NaCl (145), KCl (5.4), HEPES (10), MgCl2 (0.8), CaCl2 (1.8), Glucose (10), Sucrose (30); pH. 7.4. To block the voltage-gated sodium currents, 40 nM TTX is added in the recording solution. Intracellular solution (in mM): K-gluconate (140), HEPES (20), EGTA (1.1), Phosphocreatine (5), MgATP (3), GTP (0.3), MgCl2 (5), and CaCl2 (0.1); pH: 7.2

The whole-cell current is measured with patch-clamp amplifier (Axopatch 200B), filtered at 2 kHz, digitized at 5 kHz and recorded on a computer with pClamp 8 software. The cells are voltage-clamped at −80 mV. All compounds or saline are applied by DAD-12 system (ALA Scientific Instruments, New York).

Procedure for drug application:

10 s saline/or drug;

1 s 500 μM glutamate/or 500 μM glutamate+drug;

10 s saline

The mean value of plateau current between 600 ms to 900 ms after application of glutamate/or glutamate+drug is calculated and used as the parameter to measure the compound's effect.

B. Enhancement of AMPA Receptor Function in Acute Hippocampal Slices

The field EPSP (excitatory post-synaptic potential) recorded in field CA1 after stimulation of CA3 axons is known to be mediated by AMPA receptors, which are present in the synapses (Kessler et al., Brain Res. 560: 337-341 (1991)). Drugs that selectively block the receptor selectively block the field EPSP (duller et al., Science, supra). Aniracetam, which has been shown to increase the mean open time of the AMPA receptor channel, increases the amplitude of the synaptic current and prolongs its duration (Tang et al., Science, supra). These effects are mirrored in the field EPSP (see, for example, Staubli et al., Psychobiology, supra; Xiao et al., Hippocampus, supra; Staubli et al., Hippocampus 2: 4958 (1992)). Similar results have been reported for the previously disclosed stable benzamide analogs of aniracetam (Lynch and Rogers, PCT Pubn. No. WO 94/02475).

Hippocampal slices are maintained in a recording chamber continuously perfused with artificial cerebrospinal fluid (ACSF). During 15-30 minute intervals, the perfusion medium is switched to one containing various concentrations of the test compounds. Responses collected immediately before and at the end of drug perfusion are superimposed in order to calculate the percent increase in EPSP amplitude.

To measure the effects of test compounds, a bipolar nichrome stimulating electrode is positioned in the dendritic layer (stratum radiatum) of the hippocampal subfield CA1 close to the border of subfield CA3, as described in Example 64. Current pulses (0.1 msec) through the stimulating electrode activate a population of the Schaffer-commissural (SC) fibers, which arise from neurons in the subdivision CA3 and terminate in synapses on the dendrites of CA1 neurons. Activation of these synapses causes them to release the transmitter glutamate. Glutamate binds to post-synaptic AMPA receptors, which then transiently open an associated ion channel and permit a sodium current to enter the postsynaptic cell. This current results in a voltage in the extracellular space (the field EPSP), which is recorded by a high impedance recording electrode positioned in the middle of the stratum radiatum of CA1.

The intensity of the stimulation current is adjusted to produce half-maximal EPSPs (typically about 1.5-2.0 mV). Paired stimulation pulses are given every 40 sec with an interpulse interval of 200 msec.

The methods described above are not meant to be inclusive. For example, it is also possible to indirectly measure potentiation of AMPA receptor function by the receptor's indirect effect on internal calcium concentrations in dissociated neurons or cells of non-neuronal origin transfected with genetic material that allows expression of AMPA receptor subunits. Because of certain limitations of these indirect methods, the two methods described above are preferred.

II. Non-Human Primate Model for Testing Effects of Sleep Deprivation on a Delayed-Match-to-Sample Task (DMTS).

This task consists of a kind of “heads-up” display wherein the monkey interacts with a computer video display through motions of its hand. Stimuli are displayed on a 52-inch front projection screen using an LCD computer projector. A fluorescent spot on the back of the monkey's hand is tracked by an overhead camera, and the coordinates of the hand position are proportionally translated into movement of a cursor on the video display. The animal responds to single Sample phase images by placing the cursor inside the image, then after a delay (during which the display is blanked) the animal must select the appropriate Match image (out of 2-6 different images) by placing the cursor inside the image identical to the Sample. Animal performance is scored as correct responses according to length of delay, and complexity of the trial (number of match images). Recent findings have shown that primate hippocampal neurons encode specific features of the task and stimulus, and that strength of the encoding correlates to behavioral success. Recording and on-line analysis of hippocampal neural activity allows tracking of the animal's cognitive performance under various experimental conditions. Two additional measures (eye and limb movement tracking) along with recording of movement sensitive neurons in hippocampus, putamen and motor cortex provide assessment of attention and ability to perform directed motor movements. Thus, it is possible to distinguish behavioral errors due to inattention or slow movements from those caused by inappropriate cognitive processing of the task information.

In practice, an animal is trained in the DMTS task such that his daily performance variable is relatively constant. This is judged as baseline performance. Once a stable baseline is established, the subject is deprived of sleep for variable periods in order to establish the effect of sleep deprivation on performance. The ability of an AMPA receptor modulator to ameliorate the decline in performance in the DMTS task is assessed by administered of the test compound prior to or during testing after sleep deprivation. If the test compound is administer prior to the beginning of the test session, then the effect of the drug is compared to performance following sleep deprivation on prior or subsequent days of testing. This protocol allows for the co-administration of a metabolic tracer (such as fluorodeoxyglucose labeled with fluorine-18; FDG) so that regional brain activity can be evaluated using Positron Emission Tomography (PET). Alternatively, an intravenous administration of the test compound midway into the DMTS session allows for a within-session evaluation of drug effect. Although this protocol does not allow the use of a metabolic tracer such as FDG, it does provide a more sensitive evaluation of the effects of the test compound on the performance variable.

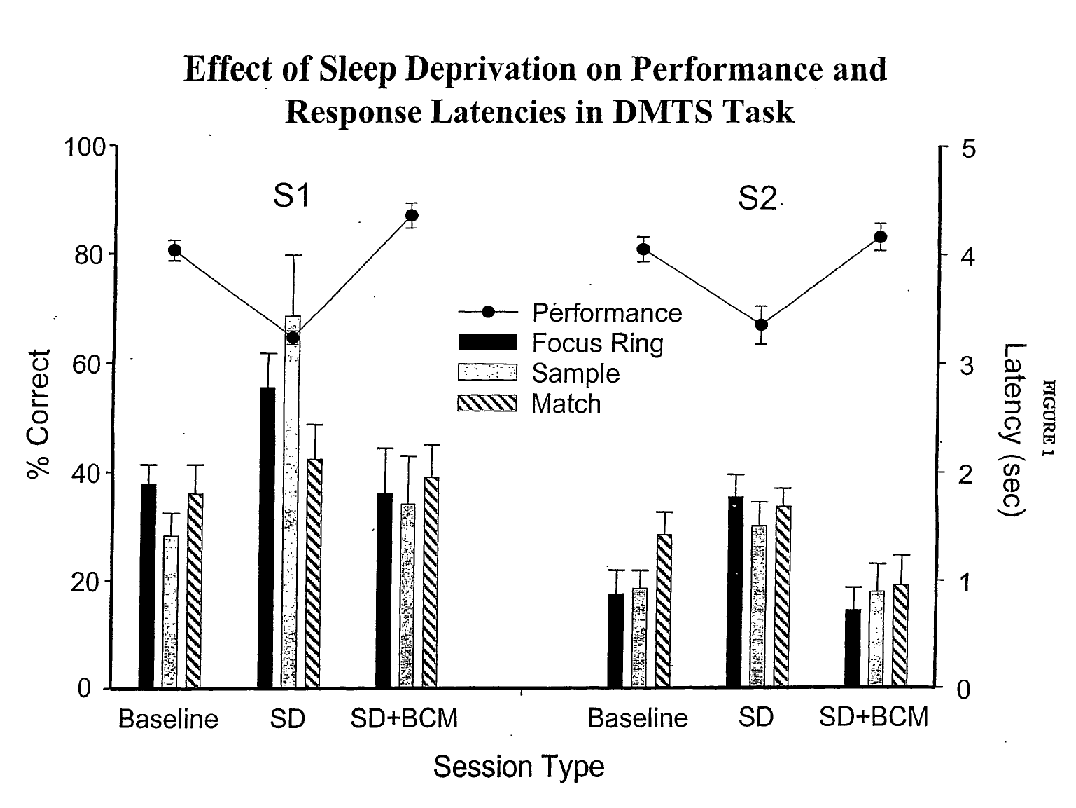

Results using the AMPA receptor modulator, 1-(benzofarazan-5-ylcarbonyl)morpholine (BCM) as the test compound in the above described protocol are shown in Table 2 and FIG. 1.

TABLE 2
Performance on Delayed Match To Sample Task

| | Performance on DMTS Task (% Correct ± sem) | | |
|---|---|---|---|
| Subject No. | Baseline | Sleep Deprivation | Sleep Deprivation + BCM |
| 1 | 81 ± 2 | 65 ± 1 | 87 ± 2 |
| 2 | 81 ± 2 | 67 ± 4 | 83 ± 3 |

A single night of sleep deprivation lowered the performance scores of both subjects by about 15 percentage points and therefore, they made nearly twice as many errors compared to baseline performance. Administration of the test compound, BCM at 0.8 mg/kg (iv) completely reversed the performance deficit, and for Subject 1, actually enhanced performance compared to baseline. Also apparent in FIG. 1 is the fact that sleep deprivation produced a significant increase in latencies to respond to the focus ring and Sample phases of the task. That this is not a general stimulant effect is supported by the observation that latencies to respond in the Match phase were not effected by drug administration. CX516 has also been shown to be effective at improving performance in this task.
III. Regional Glucose Utilization in Non-Human Primate Brain During DMTS Task

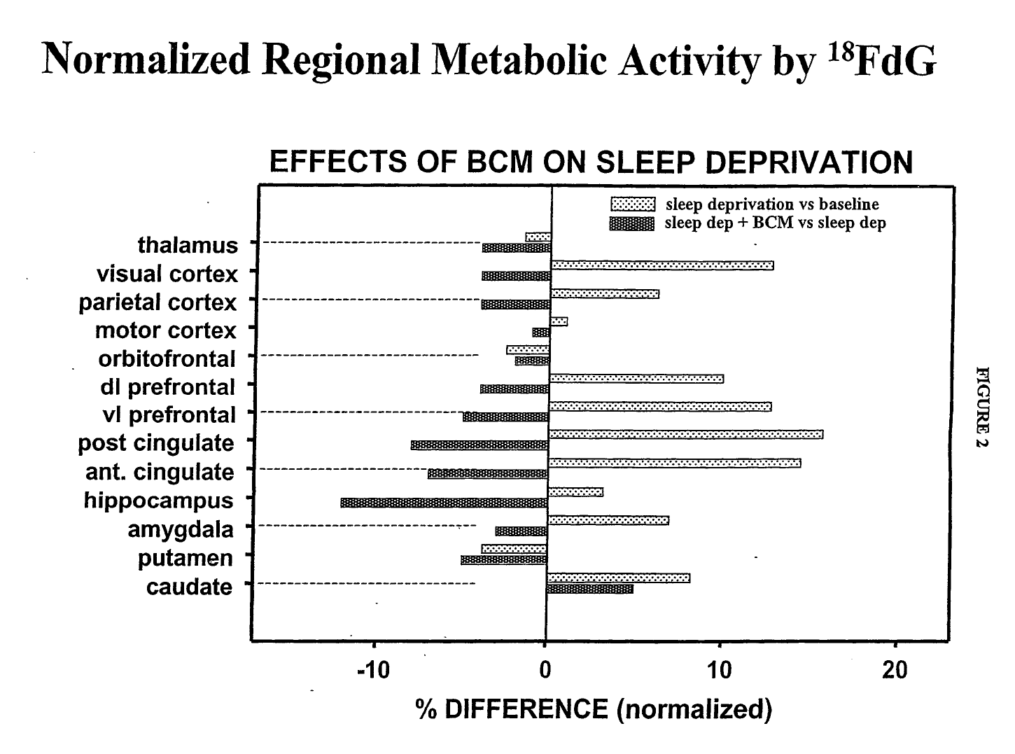

Positron Emission Tomography (PET) was used in order to examine the extent to which different brain regions were effected by sleep deprivation by measuring the uptake of ¹⁸F-labeled fluorodeoxyglucose (FDG) into cells as a measure of metabolic activity. By this method it was observed that there was a general increase in metabolism across all brain regions on test days that followed sleep deprivation compared to normal baseline test days (FIG. 2). FIG. 2 shows percent changes in absolute uptake of FDG. On test days that BCM was administered following sleep deprivation, metabolism in all brain regions was reduced compared to vehicle administration and for several regions was near normal levels (FIG. 2).

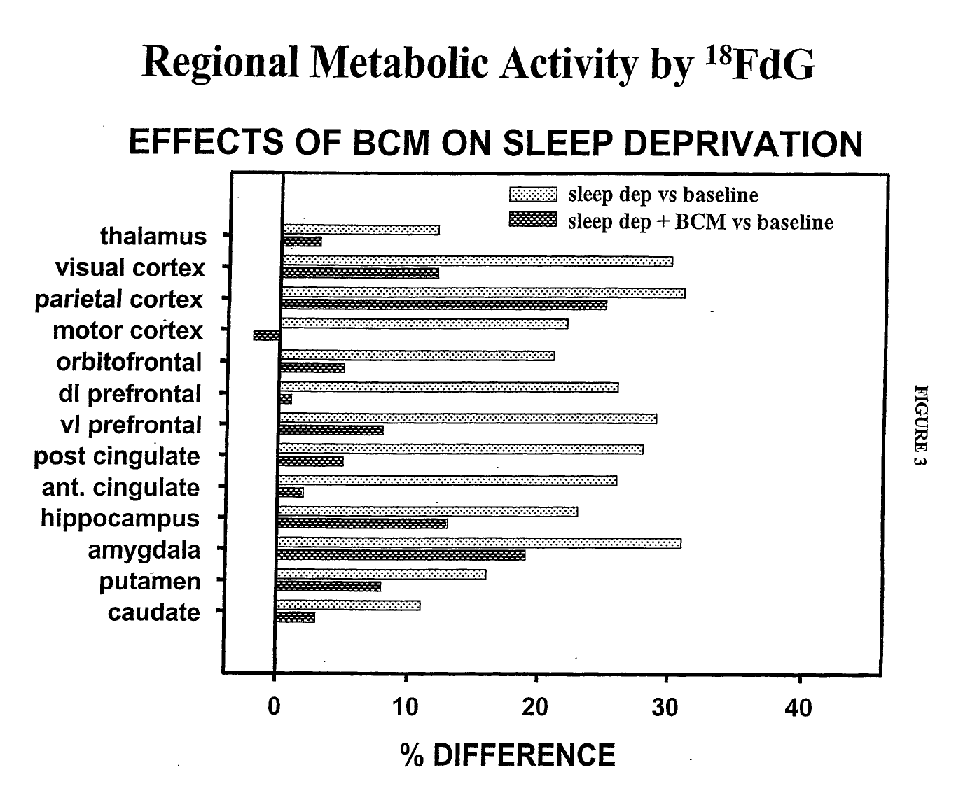

The absolute values shown in FIG. 2 can be normalized to global brain metabolism (FIG. 3) by the following equation:
(ROI_{SD}/G_{SD}−ROI_{BL}/G_{BL})/G_{BL}
where ROI mean region of interest, SD means under the condition of sleep deprivation, G means global and BL means under the condition of baseline performance. By this means of analysis, it is apparent that administration of BCM has caused a reduction in energy demand to perform the DMTS task by the subject's brain.

The description above is not meant to be limiting in regard to useful animal models of cognitive deficit induce by sleep deprivation. Other mammalian and non-mammalian subjects that can be trained to perform a memory task or whose behavior can be used to reveal a correlate of memory are also contemplated as useful in the present invention. Rodent models using water or radial arm mazes would be preferred. A primate model would be most preferred.

Salts of Compounds

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; pr (b) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Pharmaceutical Formulations

The active compounds described above may be formulated for administration in a pharmaceutical carrier in accordance with known techniques. See, e.g., Remington, The Science And Practice of Pharmacy (9th Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

Although intravenous administration was used in this test example for convenience, the formulations of the invention include those suitable for oral, rectal, topical, buccal (e.g., sublingual), vaginal, parenteral (e.g., subcutaneous, intramuscular, or intravenous), topical (i.e., both skin and mucosal surfaces, including airway surfaces) and transdermal administration, although the most suitable route in any given case will depend on the nature and severity of the condition being treated and on the nature of the particular active compound which is being used. The most preferred route would be oral.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit/dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. The compound or salt is provided in the form of a lyophilizate which is capable of being reconstituted with a suitable pharmaceutically acceptable carrier to form a liquid composition suitable for injection thereof into a subject. The unit dosage form typically comprises from about 2 to 900 mg of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis/tris buffer (pH 6) or ethanol/water and contain from 0.1 to 0.2 M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques. Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to compounds disclosed above or their salts, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

Dosage

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any one active agent, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art.

As a general proposition, a dosage from about 0.02 to about 15 mg/kg will have therapeutic efficacy, with all weights being calculated based upon the weight of the active compound, including the cases where a salt is employed. Toxicity concerns at the higher level may restrict intravenous dosages to a lower level such as up to about 5 mg/kg, with all weights being calculated based upon the weight of the active base, including the cases where a salt is employed. A dosage from about 0.1 mg/kg to about 10 mg/kg may be employed for oral administration. Typically, a dosage from about 0.5 mg/kg to 5 mg/kg may be employed for intramuscular injection. The frequency and duration of the treatment is usually once or twice per day as needed.

1. A method for treating or preventing cognitive impairment as a result of acute or chronic sleep deprivation, comprising administering to a subject or patient an effective amount of an AMPA receptor potentiator.

2. The method of claim 1 wherein the AMPA receptor potentiator is a compound according to the structure: 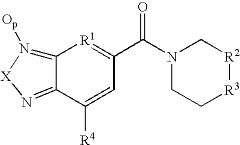
wherein X=oxygen or sulfur; R¹ is selected from the group consisting of —N═, —CR═, or —CX═; R² is selected from the group consisting of —(CRR′)ₙ—, —C(O)—, —CR═CR′—, —CR═CX—, —CRX—, —CXX′—, —S—, and —O—, and R³ is selected from the group consisting of —(CRR′)ₘ—, —C(O)—, —CR═CR′—, —CRX—, —CXX′—, —S—, and —O—; R⁴ is R or X; X and X′ are independently selected from —Br, —Cl, —F, —CN, —NO₂, —OR, —SR, —NRR′, —C(O)R, —CO₂R, or —CONRR′, wherein two groups R or R′ on an individual group X, or on two adjacent groups X, may together form a ring; and
R and R′ are independently selected from (i) hydrogen, (ii) C₁-C₆ branched or unbranched alkyl, which may be unsubstituted or substituted with one or more functionalities selected from halogen, nitro, alkoxy, hydroxy, alkylthio, amino, keto, aldehyde, carboxylic acid, carboxylic ester, or carboxylic amide, and wherein two such alkyl groups on a single carbon or on adjacent carbons may together form a ring, and (iii) aryl, which may be unsubstituted or substituted with one or more functionalities selected from halogen, nitro, alkoxy, hydroxy, aryloxy, alkylthio, amino, keto, aldehyde, carboxylic acid, carboxylic ester, or carboxylic amide;
m and p are, independently, 0 or 1; and n is 0, 1 or 2.

3. The method according to claim 1 or 2 wherein said AMPA receptor potentiator is:
1-(benzofurazan-5-ylcarbonyl)piperidine;
1-(benzofurazan-5-ylcarbonyl)-4-hydroxypiperidine;
1-(benzofurazan-5-ylcarbonyl)-4-cyanopiperidine;
1-(benzofurazan-5-ylcarbonyl)morpholine (BCM); or
1-(benzofurazan-5-ylcarbonyl)-4,4-difluoropiperidine.

3. (cancelled)

4. The method of claim 1 wherein the AMPA receptor potentiator is a compound according to the structure: 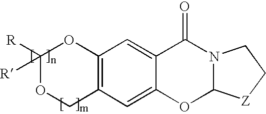
wherein
Z is —CH₂— or —O—,
R and R′ are independently hydrogen, alkyl, substituted alkyl or together form a cycloalkyl ring, or together with oxygen, sulfur or nitrogen form a heterocyclic ring.
m is 0, 1 or 2, and,
n is 1 or 2.

5. The method according to claim 1 or 4 wherein the AMPA receptor potentiator is a compound according to the chemical structure: 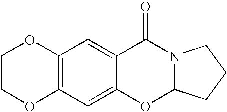
2H,3H,6aH-pyrrolidino[2″,1″-3′,2′]1,3-oxazaperhydroino[6′,5′-2,1]benzo[4,5-e]1,4-dioxin-10-one (CX614), a compound according to the chemical structure: 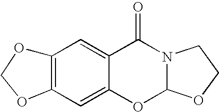
2H,7H,8H,5aH-1,3-oxazolidino[2″,3″-3′,2′]1,3-oxazaperhydroino[6′,5′-4,5]benzo[d]1,3-dioxolen-9-one (CX554), or a compound according to the chemical structure: 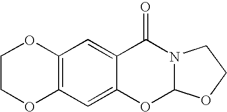
2H,3H,8H,9H,6aH-1,3-oxazolidino[2″,3″-3′,2′]1,3-oxazaperhydroino[6′,5′-4,5]benzo[e]1,4-dioxin-10-one.

6. The method of claim 1 wherein the AMPA receptor potentiator is a compound according to the structure: 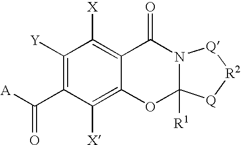
wherein
Q and Q′ are independently hydrogen, —CH₂—, —O—, —S—, alkyl, or substituted alkyl,
R¹ is hydrogen or alkyl,
R² may be absent, or if present may be —CH₂—, —CO—, —CH₂CH₂—, —CH₂CO—, —CH₂O—, —CRR′—, or —CONR—,
Y is hydrogen or —OR³, or serves to link the aromatic ring to A as a single bond, ═N— or —NR—,
R³ is hydrogen, alkyl, substituted alkyl or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

7. The method of claim 6 wherein
Q, Q′ and R² are —CH₂—,
X, X′ and R′ are hydrogen,
Y is hydrogen or —OR³, where R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

8. The method of claim 6 wherein
Q and Q′ are independently hydrogen, alkyl, or substituted alkyl,
R¹ is hydrogen or alkyl,
R² is absent,
Y is hydrogen or —OR³, or serves to link the aromatic ring to A as a single bond, ═N— or —NR—,
R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

9. The method of claim 6 wherein
Q and Q′ are independently hydrogen, alkyl, or substituted alkyl,
R¹ is hydrogen or alkyl,
R² is absent,
Y is —OR³,
R³ is a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

10. The method of claim 6 wherein
Q and Q′ are independently hydrogen, alkyl, or substituted alkyl,
R¹ is hydrogen or alkyl,
R² is absent,
Y is —OR³,
R³ is a lower alkylene such as a methylene or ethylene, or substituted lower alkylene such as —CRR′— linking the aromatic ring to A to form a substituted or unsubstituted 6, 7 or 8-membered ring,
A is —NRR′, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur, and
X and X′ are independently R, halo, —CO₂R, —CN, —NRR′, —NRCOR′, —NO₂, —N₃ or —OR.

11. The method according to claim 10 wherein said compound is:

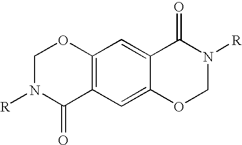

12. The method according to claim 10 wherein said compound is 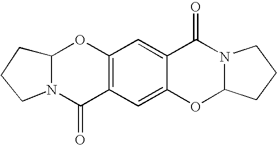
3aH,9aH-pyrrolidino[2,1-b]pyrrolidino[2″,1″-2′,3′](1,3-oxazino)[5′,6′-2,1]benzo[4,5-e]1,3-oxazaperhydroine-6,12-dione.

13. The method according to claim 1 wherein said AMPA receptor potentiator is
1-(Benzofurazan-5-ylcarbonyl)morpholine (BCM);
1-(Quinoxaline-6-ylcarbonyl)piperidine (CX516);
2H,3H,6aH-Pyrrolidino[2″,1″-3′,2′]1,3-oxazino[6′,5′-5,4]benzo[e]1,4-dioxan-10-one (CX614);
3aH,9aH-pyrrolidino[2,1-b]pyrrolidino[2″,1″-2′,3′](1,3-oxazino)[5′,6′-2,1]benzo[4,5-e]1,3-oxazaperhydroine-6,12-dione;
Aniracetam;
IDRA-21;
S18986;
PEPA;
[2-Fluoro-2-(4-{3-[(methylsulfonyl)amino]phenyl}propyl][(methylethyl)sulfonyl]amine;
N-2-(4-(3-thienyl)phenyl)propyl methanesulfonamide;
LY392098;
LY404187;
LY450108; or
LY451398.

14. A method according to any of claims 1-4 wherein cognitive impairment is a result of acute sleep deprivation.

15. A method according to any of claims 1-4 wherein cognitive impairment is a result of chronic sleep deprivation.

16. A method according to any of claims 1-4 wherein the subject is a worker whose duties cause an interruption in normal sequence or duration of sleep cycles.

17. A method according to any of claims 1-4 wherein the subject is a person suffering from circadian rhythm disruption.

18. The method according to any of claims 1-4 wherein the subject is a patient suffering from sleep disruption as a result of disease symptomatology.

19. The method according to any of claims 1-4 wherein the subject is a service animal whose performance is impaired by sleep deprivation.

20. A pharmaceutical composition for use in the treatment or prevention of cognitive impairment as a result of acute or chronic sleep deprivation in a patient or subject, comprising an effective amount of an AMPA receptor potentiator in combination with a pharmaceutically acceptable, carrier, additive or excipient.

21. The composition of claim 20 wherein the AMPA receptor potentiator is a compound according to the structure: 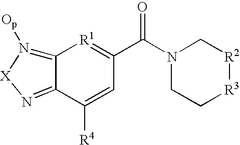
wherein X=oxygen or sulfur; R′ is selected from the group consisting of —N═, —CR═, or —CX═; R² is selected from the group consisting of —(CRR′)ₙ—, —C(O)—, —CR═CR′—, —CR═CX—, —CRX—, —CXX′—, —S—, and —O—, and R³ is selected from the group consisting of —(CRR′)ₘ—, —C(O)—, —CR═CR′—, —CRX—, —CXX′—, —S—, and —O—; R⁴ is R or X; X and X′ are independently selected from —Br, —Cl, —F, —CN, —NO₂, —OR, —SR, —NRR′, —C(O)R, —CO₂R, or —CONRR′, wherein two groups R or R′ on an individual group X, or on two adjacent groups X, may together form a ring; and
R and R′ are independently selected from (i) hydrogen, (ii) C₁-C₆ branched or unbranched alkyl, which may be unsubstituted or substituted with one or more functionalities selected from halogen, nitro, alkoxy, hydroxy, alkylthio, amino, keto, aldehyde, carboxylic acid, carboxylic ester, or carboxylic amide, and wherein two such alkyl groups on a single carbon or on adjacent carbons may together form a ring, and (iii) aryl, which may be unsubstituted or substituted with one or more functionalities selected from halogen, nitro, alkoxy, hydroxy, aryloxy, alkylthio, amino, keto, aldehyde, carboxylic acid, carboxylic ester, or carboxylic amide;
m and p are, independently, 0 or 1; and n is 0, 1 or 2.

22. The composition according to claim 20 or 21 wherein said AMPA receptor potentiator is:
1-(benzofurazan-5-ylcarbonyl)piperidine;
1-(benzofurazan-5-ylcarbonyl)-4-hydroxypiperidine;
1-(benzofurazan-5-ylcarbonyl)-4-cyanopiperidine;
1-(benzofurazan-5-ylcarbonyl)morpholine (BCM); or
1-(benzofurazan-5-ylcarbonyl)-4,4-difluoropiperidine.

23. The composition of claim 20 wherein the AMPA receptor potentiator is 1-(quinoxaline-6-ylcarbonyl)piperidine (CX516).

24. The composition of claim 20 wherein the AMPA receptor potentiator is a compound according to the structure: 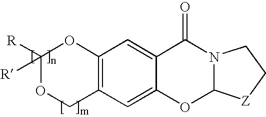
wherein
Z is —CH₂— or —O—,
R and R′ are independently hydrogen, alkyl, substituted alkyl or together form a cycloalkyl ring, or together with oxygen, sulfur or nitrogen form a heterocyclic ring.
m is 0, 1 or 2, and,
n is 1 or 2.

25. The composition according to claim 1 or 24 wherein the AMPA receptor potentiator is a compound according to the chemical structure: 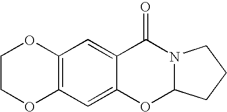
2H,3H,6aH-pyrrolidino[2″,1″-3′,2′]1,3-oxazaperhydroino[6′,5′-2,1]benzo[4,5-e]1,4-dioxin-10-one (CX614), a compound according to the chemical structure: 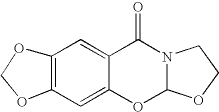
2H,7H,8H,5aH-1,3-oxazolidino[2″,3″-3′,2′]1,3-oxazaperhydroino[6′,5′-4,5]benzo[d]1,3-dioxolen-9-one (CX554), or a compound according to the chemical structure: 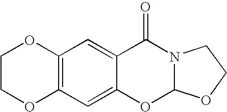
2H,3H,8H,9H,6aH-1,3-oxazolidino[2″,3″-3′,2′]1,3-oxazaperhydroino[6′,5′-4,5]benzo[e]1,4-dioxin-10-one.

26. The composition of claim 20 wherein the AMPA receptor potentiator is a compound according to the structure: 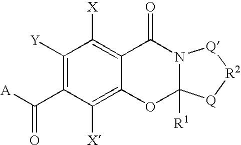
wherein
Q and Q′ are independently hydrogen, —CH₂—, —O—, —S—, alkyl, or substituted alkyl,
R¹ is hydrogen or alkyl,
R² may be absent, or if present may be —CH₂—, —CO—, —CH₂CH₂—, —CH₂CO—, —CH₂O—, —CRR′—, or —CONR—,
Y is hydrogen or —OR³, or serves to link the aromatic ring to A as a single bond, ═N— or —NR—,
R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

27. The composition of claim 26 wherein
Q, Q′ and R² are —CH₂—,
X, X′ and R¹ are hydrogen,
Y is hydrogen or —OR³, where R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

28. The composition of claim 26 wherein
Q and Q′ are independently hydrogen, alkyl, or substituted alkyl,
R¹ is hydrogen or alkyl,
R² is absent,
Y is hydrogen or —OR³, or serves to link the aromatic ring to A as a single bond, ═N— or —NR—,
R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

29. The composition of claim 26 wherein
Q and Q′ are independently hydrogen, alkyl, or substituted alkyl,
R¹ is hydrogen or alkyl,
R² is absent,
Y is —OR³,
R³ is a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —O₂R, —CN, —NRR′, —NRCOR′, —NO₂, —N₃ or —OR.

30. The composition of claim 26 wherein
Q and Q′ are independently hydrogen, alkyl, or substituted alkyl,
R¹ is hydrogen or alkyl,
R² is absent,
Y is —OR³,
R³ is a lower alkylene such as a methylene or ethylene, or substituted lower alkylene such as —CRR′— linking the aromatic ring to A to form a substituted or unsubstituted 6, 7 or 8-membered ring,
A is —NRR′, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur, and
X and X′ are independently R, halo, —CO₂R, —CN, —NRR′, —NRCOR′, —NO₂, —N₃ or —OR.

31. The composition according to claim 30 wherein said compound is:

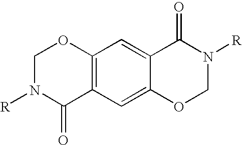

32. The composition according to claim 30 wherein said compound is 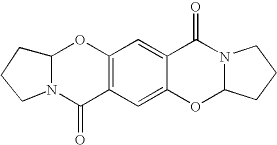
3aH,9a-H-pyrrolidino[2,1-b]pyrrolidino[2″,1″-2′,3′](1,3-oxazino)[5′,6′-2,1]benzo[4,5-e]1,3-oxazaperhydroine-6,12-dione.

33. The composition according to claim 20 wherein said AMPA receptor potentiator is:
1-(Benzofurazan-5-ylcarbonyl)morpholine (BCM);
1-(Quinoxaline-6-ylcarbonyl)piperidine (CX516);
2H,3H,6aH-Pyrrolidino[2″,1″-3′,2′]1,3-oxazino[6′,5′-5,4]benzo[e]1,4-dioxan-10-one (CX614);
3aH,9aH-pyrrolidino[2,1-b]pyrrolidino[2″,1″-2′,3′](1,3-oxazino)[5′,6′-2,1]benzo[4,5-e]1,3-oxazaperhydroine-6,12-dione;
Aniracetam;
IDRA-21;
S18986;
PEPA;
[2-Fluoro-2-(4-{3-[(methylsulfonyl)amino]phenyl}propyl][(methylethyl)sulfonyl]amine;
N-2-(4-(3-thienyl)phenyl)propyl methanesulfonamide;
LY392098;
LY404187;
LY450108; or
LY451398.

34. (cancelled)

35. The method of claim 1 wherein the AMPA receptor potentiator is 1-(quinoxaline-6-ylcarbonyl)piperidine (CX5 16).
